(19) United States
(12) Patent Application Publication
(10) Pub. No.: US 2025/0040790 A1
(43) Pub. Date: Feb. 6, 2025

(54) ELEVATORS FOR MEDICAL DEVICES

(71) Applicant: Boston Scientific Scimed, Inc., Maple Grove, MN (US)

(72) Inventors: Jordan HANAHAN, Marlborough, MA (US); Matthew Robert JAGELSKI, Milford, MA (US); Jessica FARLAND, Fiskdale, MA (US)

(73) Assignee: Boston Scientific Scimed, Inc., Maple Grove, MN (US)

(21) Appl. No.: 18/788,376

(22) Filed: Jul. 30, 2024

Related U.S. Application Data
(60) Provisional application No. 63/517,626, filed on Aug. 4, 2023.

(51) Int. Cl. A61B 1/00

(52) U.S. Cl. CPC A61B 1/00098

(57) ABSTRACT

An elevator for a medical device may comprise a proximal end portion; a distal end portion; and a guide surface for contacting an accessory device. The guide surface may extend between the proximal end portion and the distal end portion. The distal end portion may include a curved distal surface, configured such that a protruding portion of the accessory device deflects off of the distal surface as the protruding portion is moved proximally.

### CROSS-REFERENCE TO RELATED APPLICATIONS

This application claims the benefit of priority under 35 U.S.C. § 119 from U.S. Provisional Application No. 63/517,626, filed Aug. 4, 2023, which is incorporated by reference herein in its entirety.

### TECHNICAL FIELD

Various aspects of this disclosure relate generally to elevators for medical devices. In particular, aspects of this disclosure pertain to elevators having distal tips that help to prevent snagging of accessory devices or otherwise facilitate insertion and/or removal of accessory devices.

### BACKGROUND

Duodenoscopes may include a handle and a shaft, and the shaft may be insertable into a body lumen of a subject. The shaft may terminate in a distal tip portion, which may include features such as optical elements (e.g., camera, lighting), air/water outlets, and working channel openings. An elevator may be disposed at a distal tip and may be actuatable in order to change an orientation of an accessory device passed through the working channel. For example, the elevator may be pivotable or otherwise movable. During a procedure, different accessory devices may be inserted and/or removed from the working channel of the duodenoscope. Therefore, a need exists for elevators of medical devices that facilitate insertion and removal of various accessory devices.

### SUMMARY

Each of the aspects disclosed herein may include one or more of the features described in connection with any of the other disclosed aspects. Aspects of the disclosure may relate to medical device elevators that have distal tips shaped so as to help to prevent edges of accessory devices from snagging on the elevator.

In an example, an elevator for a medical device may comprise a proximal end portion; a distal end portion; and a guide surface for contacting an accessory device. The guide surface may extend between the proximal end portion and the distal end portion. The distal end portion may include a curved distal surface, configured such that a protruding portion of the accessory device deflects off of the distal surface as the protruding portion is moved proximally.

Any of the devices described herein may have any of the following features, alone or in any combination. A central portion of the distal surface may be longitudinally recessed with respect to side portions of the distal surface. The elevator may further comprise a back surface opposite the guide surface. The curved distal surface may extend between the guide surface and the back surface. A central portion of the distal surface may have a smaller thickness than side portions of the distal surface. The thickness may extend between the guide surface and the back surface. The distal surface may intersect the guide surface at an edge. A central portion of the edge may extend farther distally than side portions of the edge. The elevator may further comprise a back surface. The edge may be a first edge. The distal surface may extend between the guide surface and the back surface. The distal surface may intersect the back surface at a second edge. Side portions of the second edge may extend farther distally than a central portion of the second edge. The distal surface may have a hyperboloid shape. The distal surface may extend a uniform amount in a distal direction across a width of the distal end portion. The distal surface may be distal to an entirety of the guide surface. A central portion of the guide surface may extend further in a distal direction than side portions of the guide surface. The distal end portion may further include a ramped surface. The distal surface may be a distal end of the ramped surface. The elevator may further comprise a back surface opposite the guide surface, wherein the ramped surface tapers toward the back surface moving in a distal direction. The ramped surface may extend distally from a distal end of the guide surface. A straight line extends along an entire width of the distal surface. The distal surface may be configured to inhibit the protruding portion of the accessory device from snagging on the elevator. The distal surface may be configured to deflect off of the distal surface toward the guide surface as the protruding portion of the accessory device is moved proximally.

In another example, an elevator for a medical device may comprise a proximal end portion; a distal end portion; and a guide surface for contacting an accessory device. The guide surface may extend between the proximal end portion and the distal end portion. The distal end portion may include a curved distal surface. The curved distal surface may be thinner at a central portion of the curved distal surface than on sides of the curved distal surface.

Any of the devices disclosed herein may have any of the following features, alone or in any combination. The central portion of the curved distal surface may be proximal of the sides of the curved distal surface.

In a further example, an elevator for a medical device may comprise a guide surface for contacting an accessory device; a back surface, opposite the guide surface; and a ramped distal surface extending distally from the guide surface toward the back surface. A distalmost edge of the ramped distal surface may be distal to an entirety of the guide surface.

Any of the devices disclosed herein may have any of the following features, alone or in any combination. A straight line may extend along the distalmost surface, between a first side of the distalmost edge and a second side of the distalmost edge. The distalmost edge may be approximately perpendicular to a longitudinal axis of the guide surface.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A and 1B depict an exemplary medical device.

FIGS. 2A-2F depict aspects of an exemplary elevator for use with the medical device of FIGS. 1A and 1B.

FIGS. 3A-3G depict aspects of another exemplary elevator for use with the medical device of FIGS. 1A and 1B.

FIGS. 4A-4D depict aspects of a further exemplary elevator for use with the medical device of FIGS. 1A and 1B.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. Some Figures include arrows labeled “P” and “D” to indicate proximal and distal directions, respectively. The term “exemplary” is used in the sense of “example,” rather than “ideal.” The term “approximately,” or like terms (e.g., “substantially”), includes values+/−10% of a stated value.

Medical devices, such as duodenoscopes, may include elevators that are used to, for example, change an orientation of one or more accessory medical devices. Such elevators may be utilized with medical devices that are side-viewing (have imaging and/or lighting elements that face substantially radially outward) or forward-facing (having imaging and/or lighting elements that face substantially distally). An accessory medical device may include one or more protruding portions that may snag or otherwise interact or interfere with an edge of some conventional elevators, particularly as the accessory medical device is withdrawn proximally from the medical device. The elevators disclosed herein may have distal portions that inhibit accessory devices from snagging/getting caught on the elevator. In aspects, an elevator may have a curved distal edge that helps an accessory device to move proximally along the elevator without protrusions of the accessory device getting caught on an edge of the elevator. In other aspects, an elevator may include a ledge that has a surface that helps an accessory device to move proximally along the elevator without protrusions of the accessory device getting caught on an edge of the elevator.

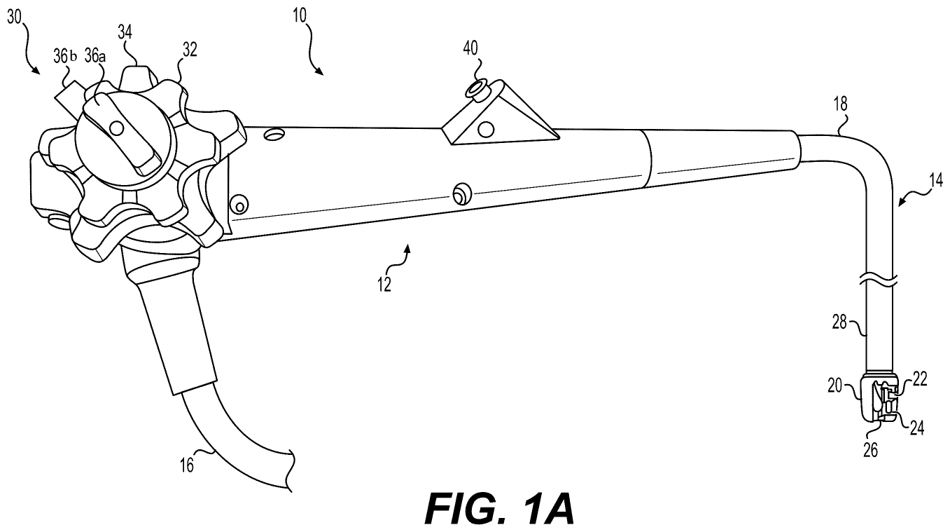

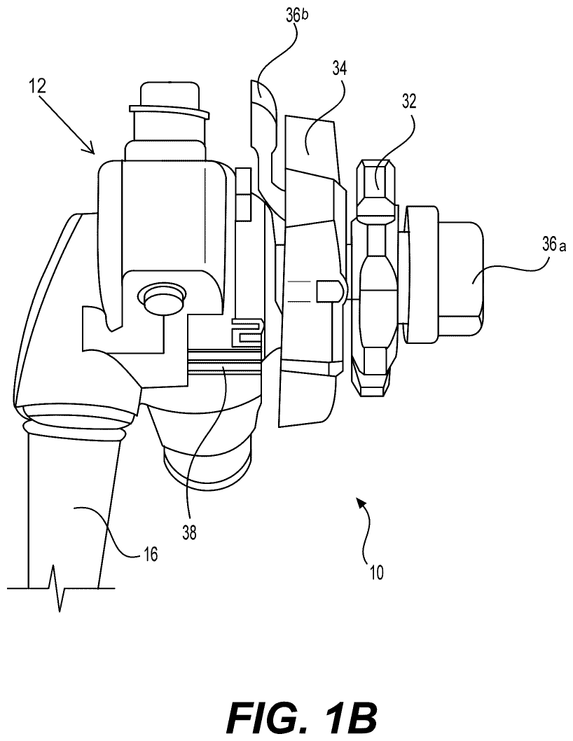

FIG. 1A depicts an exemplary medical device 10 having a handle 12 and an insertion portion 14. FIG. 1B shows a proximal end of handle 12. Medical device 10 may also include an umbilicus 16 for purposes of connecting medical device 10 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a sheath or shaft 18 and a distal tip 20. Distal tip 20 may include an imaging device 22 (e.g., a camera) and a lighting source 24 (e.g., an LED or an optical fiber). Distal tip 20 may be side-facing. That is, imaging device 22 and lighting source 24 may face radially outward, perpendicularly, approximately perpendicularly, or otherwise transverse to a longitudinal axis of shaft 18 and distal tip 20. Additionally or alternatively, distal tip 20 may include one or more imaging devices 22 that face in more than one direction. For example, a first imaging device 22 may face radially outward, and a second imaging device 22 may face distally (approximately parallel to a longitudinal axis of distal tip 20/shaft 18).

Distal tip 20 may also include an elevator 26 for changing an orientation of an accessory device or a tool inserted in a working channel of medical device 10. Elevator 26 may alternatively be referred to as a swing stand, pivot stand, raising base, or any suitable other term. Elevator 26 may be pivotable via, e.g., an actuation wire or another control element that extends from handle 12, through shaft 18, to elevator 26.

A distal portion of shaft 18 that is connected to distal tip 20 may have a steerable section 28. Steerable section 28 may be, for example, an articulation joint. Shaft 18 and steerable section 28 may include a variety of structures that are known or may become known in the art.

Handle 12 may have one or more actuators/control mechanisms 30. One or more of control mechanisms 30 may provide control over steerable section 28. One or more of control mechanisms may allow for provision of air, water, suction, etc. For example, handle 12 may include control knobs 32, 34 for left, right, up, and/or down control of steerable section 28. For example, one of knobs 32, 34 may provide left/right control of steerable section 28, and the other of knobs 32, 34 may provide up/down control of steerable section 28. Handle 12 may further include one or more locking mechanisms 36a, 36b (e.g., knobs or levers) for preventing steering and/or braking of steerable section 28 in at least one of an up, down, left, or right direction. Handle 12 may include an elevator control lever 38 (see FIG. 1B). Elevator control lever 38 may raise and/or lower elevator 26, via one or more connections between lever 38 and elevator 26. For example, in some aspects an actuating wire (not shown) extends from lever 38, through at least a portion of handle 12 and through shaft 18, to elevator 26. A port 40 may allow passage of a tool through port 40, into a working channel of medical device 10, through shaft 18, to distal tip 20. The working channel (not shown) may extend from port 40, through shaft 18, and to distal tip 20.

In use, an operator may insert at least a portion of shaft 18 into a body lumen of a subject. Distal tip 20 may be navigated to a procedure site in the body lumen. The operator may insert an accessory device (not shown) into port 40, and pass the accessory device through shaft 18 via the working channel to distal tip 20. The accessory device may exit the working channel at distal tip 20. The user may use elevator control lever 38 to raise elevator 26 and angle the accessory device toward a desired location (e.g., a papilla of the pancreatico-biliary tract). The user may use the accessory device to perform a medical procedure. Exemplary accessory devices may include snares, staplers, graspers, catheters, tomes, stents, clips, balloons, baskets, forceps, knives, electrodes, cholangioscopes, or needles.

As discussed above, although FIG. 1A depicts a side-viewing medical device 10 (a device having imaging device 22 and/or lighting source 24 facing substantially radially outward), it will be appreciated that medical device 10 may alternatively be a forward-viewing device (a device having imaging device 22 and/or lighting source 24 facing substantially distally). In aspects, medical device 10 may include both a side-viewing imaging device 22 and/or lighting source 24 and a forward-facing imaging device 22 and/or lighting source 24.

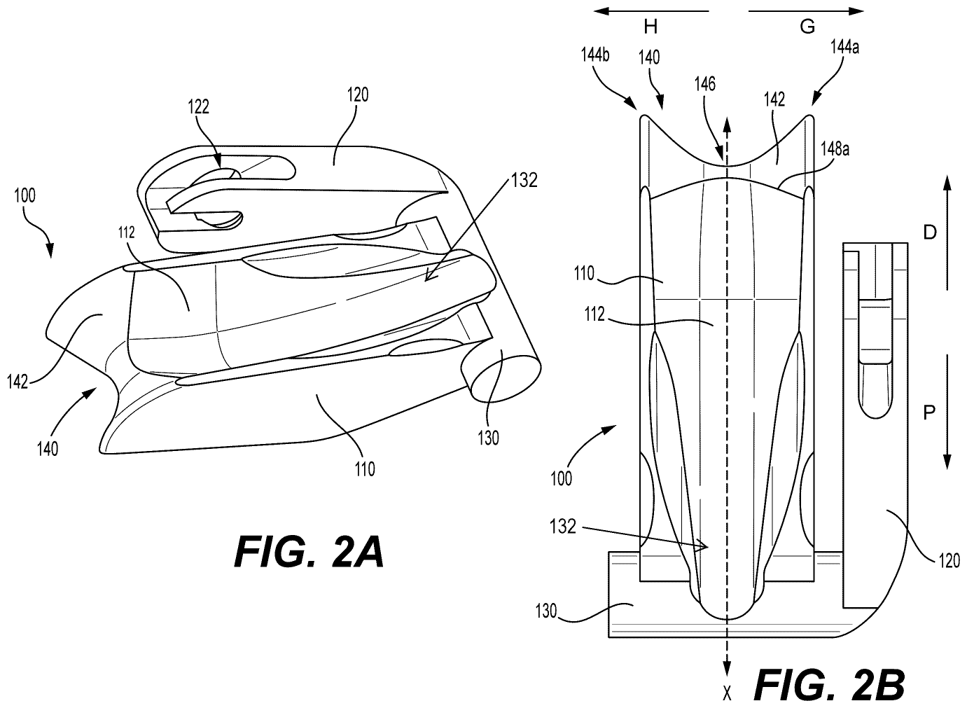

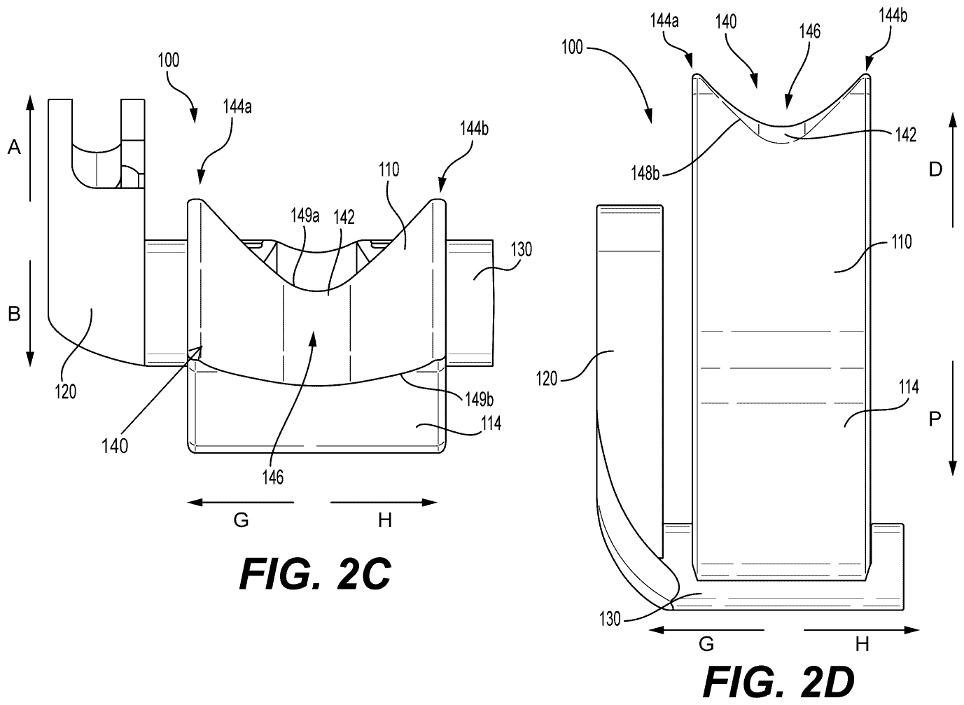

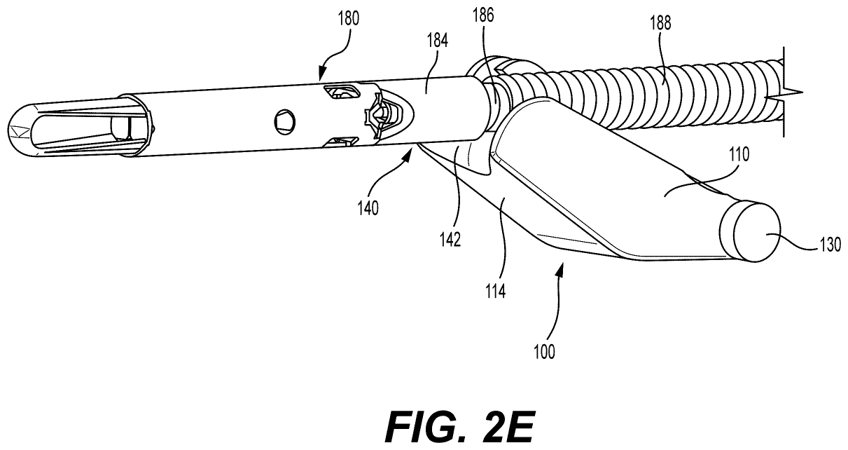

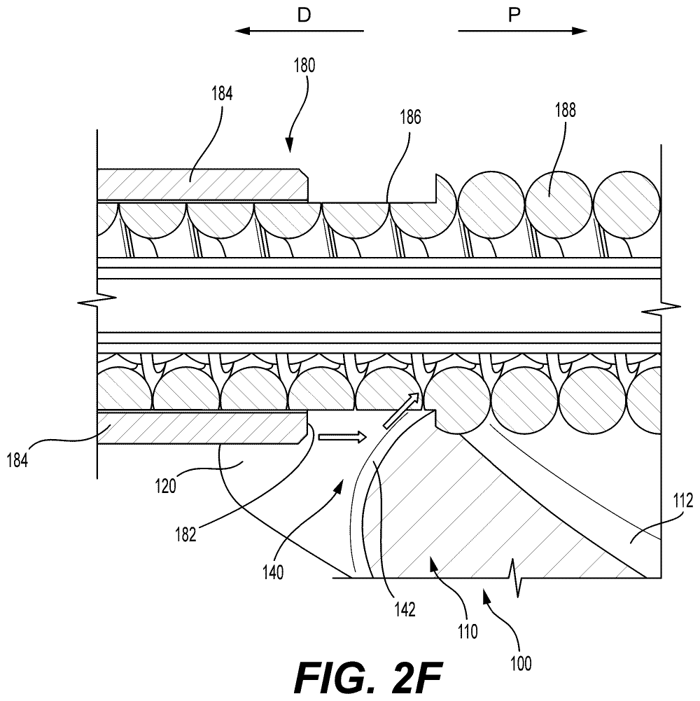

FIGS. 2A-2F depict features of an elevator 100, which may have any of the features of elevator 26 and may be used in conjunction with medical device 10. FIG. 2A shows a perspective view of elevator 100. FIG. 2B shows a plan view of a front of elevator 100. FIG. 2C shows a distal end of elevator 100. FIG. 2D shows a plan view of a back of elevator 100. FIG. 2E shows a perspective view of elevator 100 interacting with an accessory device 180. FIG. 2F shows a cross-sectional view of a portion of elevator 100 interacting with accessory device 180.

As shown particularly in FIGS. 2A-2D, elevator 100 may include a body 110 and an arm 120. Arm 120 may include a coupler 122 (e.g., an opening, slot, and/or other structure), labeled in FIG. 2A, for coupling a control wire (not shown) to arm 120. As the control wire is moved or urged distally or proximally, elevator 100 may rotate about an axle 130. Axle 130 may be at a proximal end of elevator 100 (e.g., a proximalmost end of elevator 100), coupled to a proximal end portion 132 of body 110. The arrangement above is merely exemplary.

Body 110 may have a guide surface 112 (FIGS. 2A and 2B) on a front of elevator 100 (a side of elevator 100 facing a distal opening of a working channel of medical device 10). Guide surface 112 may extend between proximal end portion 132 and a distal portion of the elevator. Guide surface 112 may contact or otherwise interact with an accessory device 180 (see FIGS. 2E and 2F) in order to direct accessory device 180. Elevator 100 may be rotated with respect to distal tip 20 (FIG. 1A) in order to raise (close) and lower (open) elevator 100. As elevator 100 raises and lowers, elevator 100 (e.g., guide surface 112) may change an orientation of accessory device 180. Body 110 may also have a back surface 114, on an opposite side of body 110 from guide surface 112. Back surface 114 may face a housing of distal tip 20.

Elevator 100 may have a distal end 140. As described in further detail below, distal end 140 of elevator 100 may have a rounded shape that deflects protruding portions of accessory devices, such as accessory device 180, so that the accessory devices do not snag on distal end 140. As shown in FIGS. 2B-2D, distal end 140 may extend between a first side 144a (closer to arm 120) of body 110 and a second side 144b (farther from arm 120) of body 110. For ease of discussion, direction G is labeled on some of the Figures to show a direction toward first side 144a. Direction H is labeled on some of the Figures to show a direction toward second side 144b.

Distal end 140 may extend further in a distal direction on first side 144a and second side 144b than in a central portion 146 of body 110 (at or near a central longitudinal axis X of body 110, labeled in FIG. 2B). In other words, distal end 140 may be recessed at central portion 146 of body 110 relative to sides 144a, 144b. As shown in FIG. 2B, when elevator 100 is viewed with guide surface 112 facing up, distal end 140 may have an approximately arcuate shape, such that distal end 140 has a concave shape. Although FIG. 2B shows distal end 140 as having a shape similar to that of a vertex of an ellipse, it will be appreciated that distal end 140 may have any suitable shape (e.g., a portion of a circle or an alternative curved shape).

As shown particularly in FIGS. 2A and 2E, distal end 140 may have a curved surface 142 that extends between guide surface 112 and back surface 114. Curved surface 142 may be symmetrical about a plane that extends along central longitudinal axis X and is perpendicular to the G/H directions. Curved surface 142 may have a curved cross-section (e.g., a shape that is approximately a circular arc) along that plane. As shown in FIG. 2B, curved surface 142 may intersect guide surface 112 at an edge 148a. Edge 148a may have an arcuate shape when viewed with guide surface 112 facing up. A central portion of edge 148a (near central portion 146 of body 110) may be distal to side portions of edge 148a near sides 144a, 144b of body 110. As shown in FIG. 2D, curved surface 142 may intersect back surface 114 at an edge 148b. Edge 148b may have an arcuate shape. A central portion of edge 148b (near central portion 146 of body 110) may be proximal to side portions of edge 148b near sides 144a, 144b of body 110. Thus, edges 148a and 148b may curve in opposite directions form one another.

As best shown in FIG. 2C, curved surface 142 may be thinner (in a direction labeled A/B for convenience, which is perpendicular to proximal/distal directions and G/H directions) near central portion 146 of body 110. Direction A may be a direction toward guide surface 112 (not labeled in FIG. 2C) and away from back surface 114. Direction B may be a direction toward back surface 114 and away from guide surface 112. Curved surface 142 may be thicker near sides 144a, 144b of body 110. Stated another way, curved surface 142 may have a smaller thickness near central portion 146 than near sides 144a, 144b. In other words, curved surface 142 may extend higher (in direction A) at or near sides 144a, 144b of body 110 than near central portion 146. When viewing a distal end of elevator 100 (as shown in FIG. 2C), curved surface 142 may have an arcuate upper perimeter 149a and an arcuate lower perimeter 149b. Upper perimeter 149a may have a steeper curve than lower perimeter 149b. In some examples, upper perimeter 149a may be edge 148a, and/or lower perimeter 149b may be edge 148b. In other examples, upper perimeter 149a may differ from edge 148a and/or lower perimeter 149b may differ from edge 148b.

Curved surface 142 may have a shape that is similar to a portion of a surface of a hyperboloid or an hourglass. Where the term “hyperboloid” or “hourglass” is used herein, it will be appreciated that the term encompasses shapes that are substantially similar to those shapes. It will also be appreciated that reference to a hyperboloid or hourglass shape includes a portion of a hyperboloid or an hourglass.

FIGS. 2E and 2F show elevator 100 interacting with accessory device 180. Accessory device 180 is depicted as being a clip, but accessory device 180 may be any type of accessory device (e.g., snare, basket, knife, needle, suturing device, guidewire, tome, stent insertion device, ligation device, etc.). Accessory device 180 may include protruding portions. For example, accessory device 180 may include a proximal edge 182 (see FIG. 2F) of a sheath 184. Sheath 184 may partially cover a first coil 186. As shown in FIG. 2F, coil 186 may include wires that are not round (e.g., that have a flat radially outer surface). Accessory device 180 may also include a second coil 188 that includes round wires. As shown in FIG. 2F, coils 186, 188 may be formed from a same piece of wire, with portions of the wire removed to form first coil 186. Proximal edge 182 may protrude relative to first coil 186, which is adjacent to proximal edge 182. The properties of accessory device 180, discussed above, are merely exemplary. Various types of accessory devices may have various structures that may have various protruding elements or other features.

As shown particularly in FIG. 2F, when proximal edge 182 of sheath 184 contacts curved surface 142 of elevator 100 (e.g., as accessory device 180 is being withdrawn in a proximal direction), curved surface 142 may help to deflect proximal edge 182 as shown in the arrows of FIG. 2F. Thus, proximal edge 182 may not snag on curved surface 142 or any portion of distal end 140 of elevator 100. Elevator 100 may lack edges on which proximal edge 182 may snag. Edge 148a may be gradual/smooth enough and located in a position where proximal edge 182 may not snag, get caught, etc. Curved surface 142 may lack corners and may define a large opening for receiving accessory device 180, as shown particularly in FIG. 2C. Thus, elevator 100 may be designed so as to increase ease of removing accessory devices 180 or otherwise moving accessory devices 180 proximally during a procedure. In contrast, conventional elevators may include edges on which proximal edge 182 of accessory device 180 (or other protruding portions of accessory device 180) may snag or otherwise become caught. Such snags may increase procedure time or difficulty or may damage device 180.

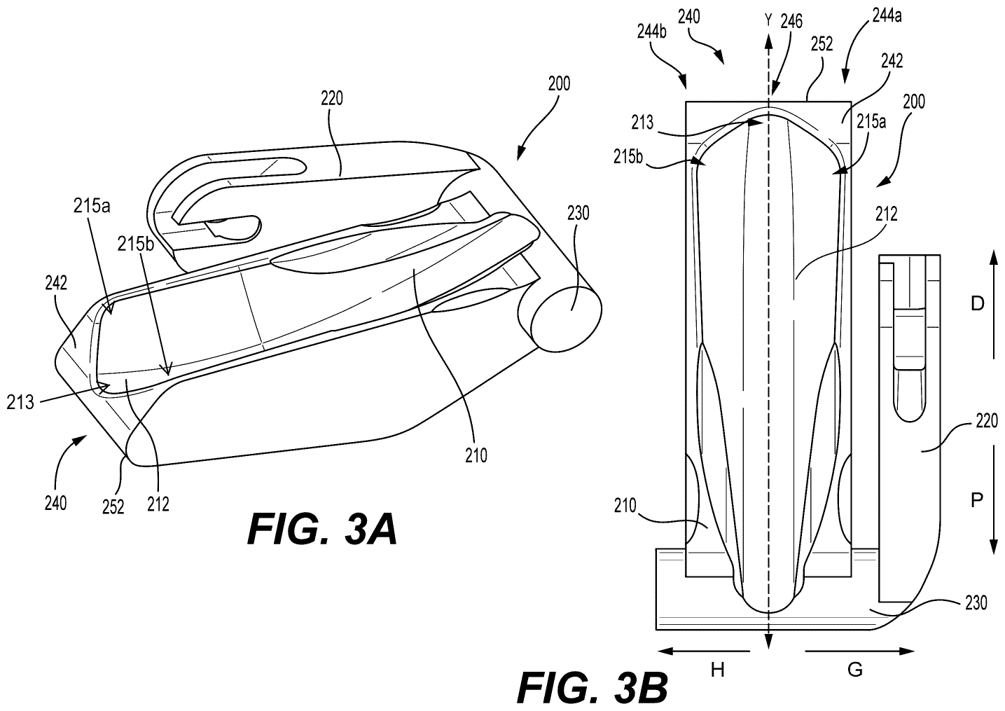

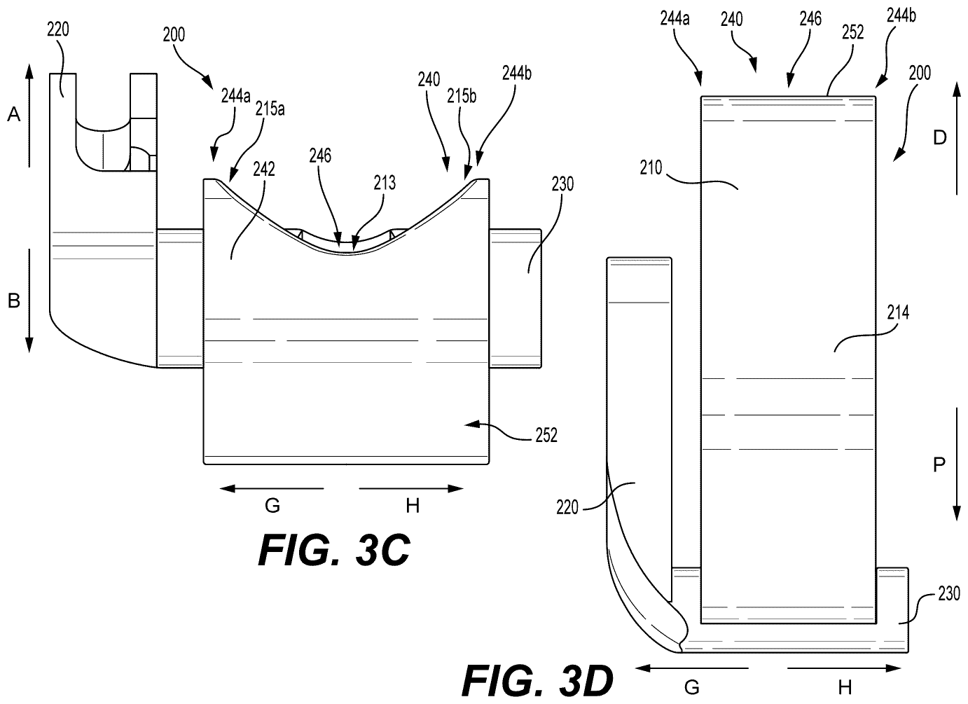

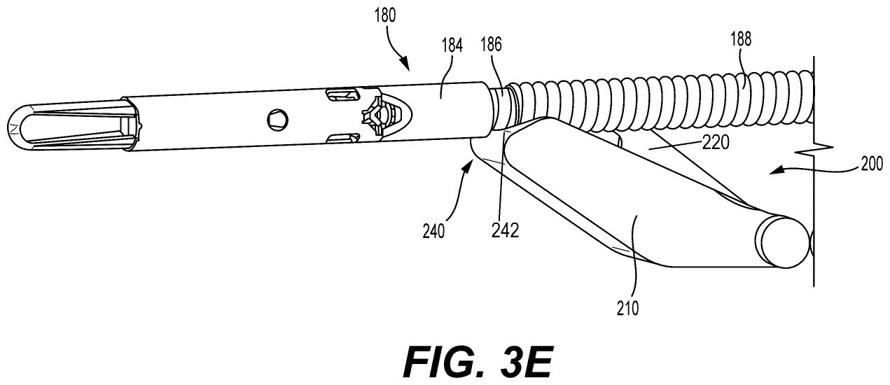

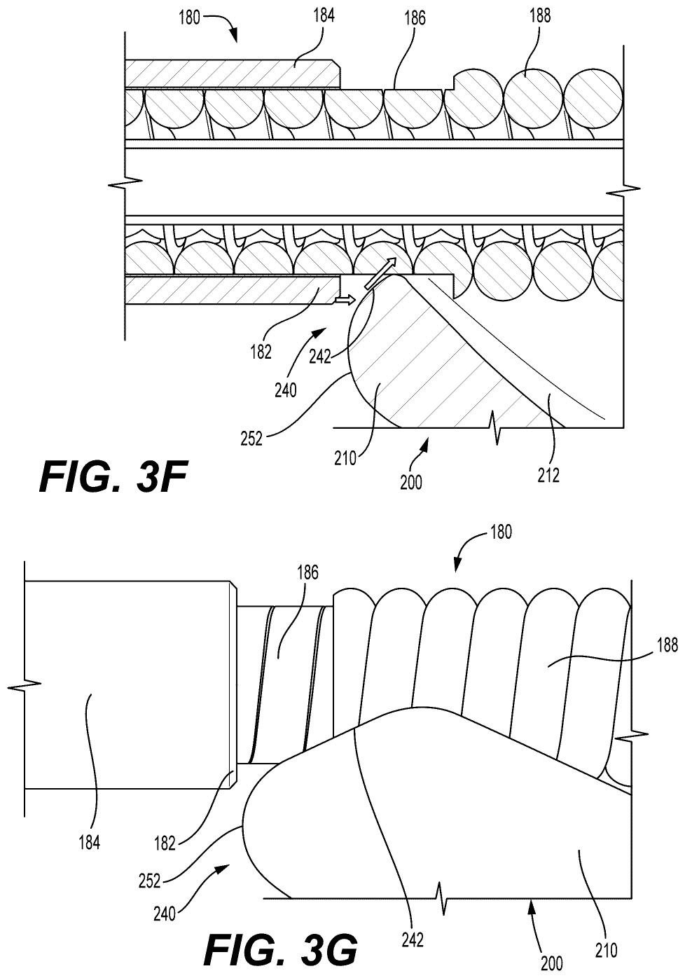

FIGS. 3A-3G depict an alternative elevator 200. Except as specifically described herein, elevator 200 may have any of the properties of elevators 26, 100, above. Similarly, elevator 100 may have properties of elevator 200, unless otherwise specified above. Where feasible, the reference numbers of FIGS. 3A-3G add 100 to the reference numbers of FIGS. 2A-2F to denote similar structures. FIG. 3A shows a perspective view of elevator 200. FIG. 3B shows a plan view of a front of elevator 200. FIG. 3C shows a distal end of elevator 200. FIG. 3D shows a plan view of a back of elevator 200. FIG. 3E shows a perspective view of elevator 200 interacting with accessory device 180. FIG. 3F shows a cross-sectional view of a portion of elevator 200 interacting with accessory device 180. FIG. 3G shows a side view of a portion of elevator 200 interacting with accessory device 180.

As with elevator 100, elevator 200 may include a body 210 (having any of the features of body 110) and an arm 220 (having any of the features of arm 120). Elevator 200 may further include an axle 230, having any of the properties of axle 130.

Elevator 200 may have a distal end 240. As described in further detail below, distal end 240 of elevator 200 may have a ledge shape that deflects protruding portions of accessory devices, such as accessory device 180, so that the accessory devices do not snag on distal end 240. As shown in FIG. 3B, distal end 240 may extend between a first side 244a of body 210 and a second side 244b of body 210. For ease of discussion, direction G is labeled on some of the Figures to show a direction toward first side 244a. Direction H is labeled on some of the Figures to show a direction toward second side 244b. As shown in FIG. 3B, when elevator 200 is viewed with a guide surface 212 (having any of the properties of guide surface 112) facing up, distal end 240 may have an approximately straight shape. Likewise, as shown in FIG. 3D, distal end 240 may have an approximately straight shape when viewed with a back surface 214 (having any of the properties of back surface 214) facing up. As shown in FIGS. 3B and 3D, distal end 240 may form a straight line that extends approximately parallel to the G/H directions and approximately perpendicularly to a longitudinal axis Y of body 210. Stated a different way, distal end 240 may extend an approximately uniform amount in a distal direction, across an entire width of distal end 240.

As shown in FIGS. 3A and 3B, guide surface 212 may have a tapered distal end, such that a central portion 213 of guide surface 212 (closer to a central longitudinal axis Y of body 210 or a central portion 246 of body 210) extends farther distally than side portions 215a, 215b of guide surface 212 (closer to first side 244a and second side 244b, respectively). As shown in FIG. 3C, central portion 213 of guide surface 212 (at central portion 246 of body 210) may be recessed in an A/B direction (along a height of elevator 200) relative to side portions 215a, b of guide surface 212. Thus, a shape of guide surface 212 may help to retain accessory device 180 on guide surface 212, without accessory device 180 falling off of sides 244a, 244b of body 210.

As shown particularly in FIGS. 3A and 3E, distal end 240 may have a ramped surface 242 that extends between guide surface 212 and back surface 214 (FIG. 3D). Ramped surface 242 may be symmetrical about a plane extending along a central longitudinal axis Y of body 210 (labeled in FIG. 3B), parallel to proximal/distal directions and perpendicular to the G/H directions. Ramped surface 242 may extend toward back surface 214 from first side 244a, second side 244b, and guide surface 212. Ramped surface 242 may have a tapered height (in an A/B direction, labeled in FIG. 3C), sloping downward (in the B direction, toward back surface 214) moving distally along ramped surface 242. As shown in FIG. 3A, proximal portions of ramped surface 242 may be approximately planar.

At a distal end of ramped surface 242, ramped surface 242 may curve as it meets back surface 214, so as to form a curved distalmost edge 252 rather than a sharp edge. Distalmost edge 252 may extend approximately perpendicularly to longitudinal axis Y, such that a straight line may extend along an entire width of distalmost edge. In cross-section (along a plane along central longitudinal axis Y and perpendicular to directions G/H), curved distalmost edge 252 may have a similar cross-sectional shape to a curved surface 142, as shown by comparing FIGS. 2F and 3F.

As discussed above, and a shown in FIGS. 3B and 3D, distalmost edge 252 may be approximately straight, across a width of elevator 200 (approximately perpendicular to longitudinal axis Y). Distalmost edge 252 may be distal of an entirety of guide surface 212. A distance between distalmost edge 252 and guide surface 212 may be greater near central portion 213 of guide surface than side portions 215a, b of guide surface, because central portion 213 extends farther distally than side portions 215a, 215b.

FIGS. 3E-3G show elevator 200 interacting with accessory device 180 (having the properties discussed above). As shown particularly in FIGS. 3F and 3G, when proximal edge 182 of sheath 184 contacts a portion of ramped surface 242, such as distalmost edge 252 (e.g., as accessory device 180 is being withdrawn in a proximal direction), ramped surface 242 may help to deflect proximal edge 182 as shown in the arrows of FIG. 3F. In examples, proximal edge 182 may contact a portion of elevator 200 where distalmost edge 252 and ramped surface 242 meet. Proximal edge 182 may not snag on ramped surface 242 or any portion of distal end 240 of elevator 200. Elevator 200 may lack edges on which proximal edge 282 may snag. Ramped surface 242 may lack corners and may define a large opening for receiving accessory device 180, as shown particularly in FIG. 3C. Thus, elevator 200 may be designed so as to increase ease of removing accessory devices 180 during a procedure. Conventional elevators may include edges on which proximal edge 182 of accessory device 180 (or other protruding portions of accessory device 180) may snag or otherwise become caught. Such snags may increase procedure time or difficulty or may damage accessory device 180.

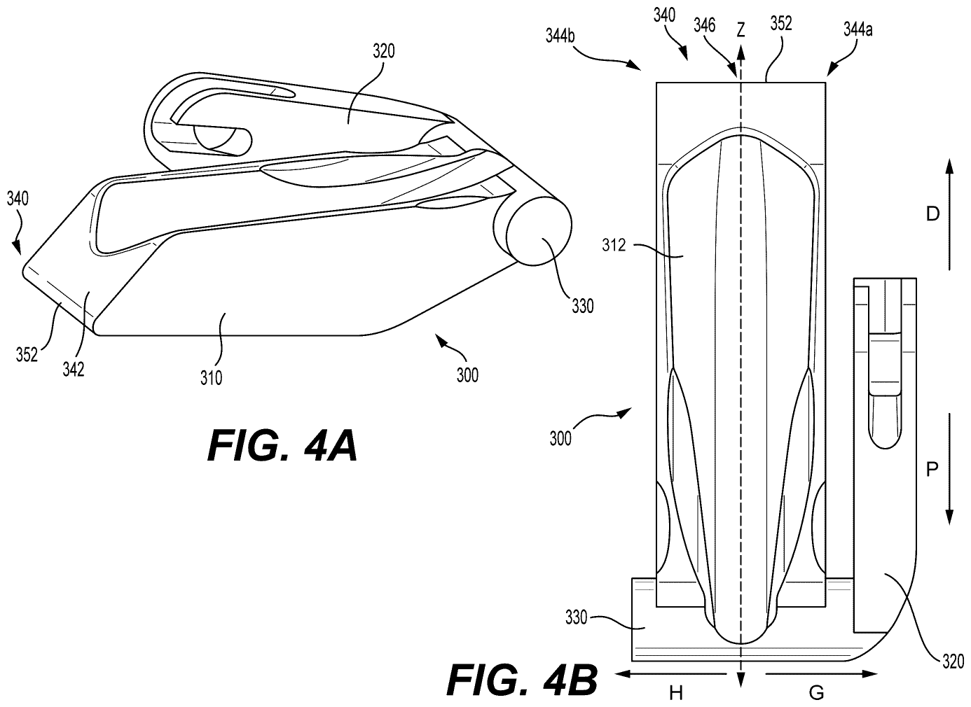

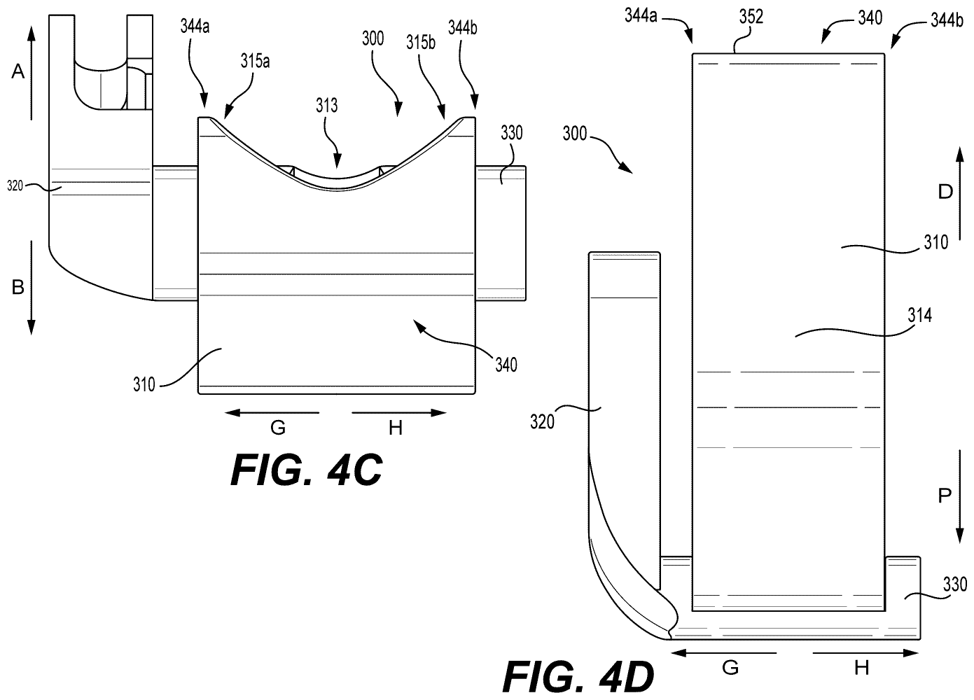

FIGS. 4A-4D depict an alternative elevator 300. Elevator 300 may have any of the properties of elevators 26, 100, 200, unless specifically provided below. Where feasible, the reference numbers of FIGS. 4A-4D add 100 to the reference numbers of FIGS. 3A-2G to denote similar structures. FIG. 4A shows a perspective view of elevator 300. FIG. 4B shows a plan view of a front of elevator 300. FIG. 4C shows a distal end of elevator 300. FIG. 4D shows a plan view of a back of elevator 300.

As with elevators 100, 200 elevator 300 may include a body 310 (having any of the features of bodies 110, 210) and an arm 320 (having any of the features of arms 120, 220). Elevator 200 may further include an axle 330, having any of the properties of axles 130, 230. Elevator 300 may be similar to elevator 200, but may have a distal end 340 with a ramped surface 342 that extends farther distally than ramped surface 242 of distal end 240. As shown in FIG. 4B, guide surface 312 may have a tapered distal end, as discussed above for guide surface 212.

Elevator 300 may have distal end 340. As described in further detail below, distal end 340 of elevator 300 may have a ledge shape that deflects protruding portions of accessory devices, such as accessory device 180 (depicted and described with respect to elevators 100, 200), so that the accessory devices do not snag on distal end 340. As compared with distal end 240, the ledge shape of distal end 340 may extend farther distally and, thus, may be larger than the ledge of distal end 240. As shown in FIG. 4B, distal end 340 may extend between a first side 344a of body 310 and a second side 344b of body 310. For ease of discussion, direction G is labeled on some of the Figures to show a direction toward first side 344a. Direction H is labeled on some of the Figures to show a direction toward second side 344b.

As shown in FIG. 4B, when elevator 300 is viewed with a guide surface 312 (having any of the properties of guide surfaces 112, 212) facing up, distal end 340 may have an approximately straight shape, similar to distal end 240. Likewise, as shown in FIG. 4D, distal end 340 may have an approximately straight shape when viewed with a back surface 314 (having any of the properties of back surfaces 114, 214) facing up, similar to distal end 240. As shown in FIGS. 4B and 4D, distal end 340 may form a straight line that extends approximately parallel to the G/H directions. In other words, distal end 340 may extend an approximately uniform amount in a distal direction, across an entire width of distal end 340. In other words, a portion of distal end near a central portion 346 of body 310 may extend approximately a same amount distally as a portion of distal end near sides 344a, 344b. As compared with body 210, body 310 may have a greater longitudinal length along longitudinal axis Z, due to an increased length of the ledge of distal end 340.

As shown particularly in FIG. 4A, distal end 340 may have a ramped surface 342 that extends between guide surface 312 and back surface 314 (FIG. 4D). Ramped surface 342 may be similar to ramped surface 242 but may extend farther distally than ramped surface 242 and/or may be longer in a longitudinal dimension than ramped surface 242. Ramped surface 342 may be symmetrical about a plane that extends along a central longitudinal axis Z of body 310 and perpendicular to the G/H directions. Ramped surface 342 may extend toward back surface 314 from first side 344a, second side 344b, and guide surface 312. As with ramped surface 242, ramped surface 342 may have a tapered height (in an A/B direction, labeled in FIG. 4C), sloping downward (in the B direction) moving distally along ramped surface 342. A slope of ramped surface 342 may be more gradual than a slope of ramped surface 242, because of a greater relative length of ramped surface 342. As shown in FIG. 4A, proximal portions of ramped surface 342 may be approximately planar (may be straight rather than curved).

Ramped surface 342 may curve as it meets back surface 314, so as to form a curved distalmost edge 352 rather than a sharp edge. Curved distalmost edge 352 may be a curved surface. As discussed above for distalmost edge 252, distalmost edge 352 may have a cross-sectional shape (along a plane along central longitudinal axis Z and perpendicular to directions G/H) that is similar to that of curved surface 142, as shown by comparing FIGS. 2F and 3F.

As discussed above, and a shown in FIGS. 4B and 4D, distalmost edge 352 may be approximately straight, across a width of elevator 300 (approximately perpendicular to longitudinal axis Y). Distalmost edge 352 may be distal of an entirety of guide surface 312. A distance between distalmost edge 352 and guide surface 312 may be greater near a central portion 313 of guide surface than side portions 315a, 315b of guide surface, because central portion 313 extends farther distally than side portions 315a, 315b. A distance between distalmost edge 352 and guide surface 312 may be greater than the distance between distalmost edge 252 and guide surface 212 of elevator 200.

Elevator 300 may interact with accessory device 180 similarly to how elevator 200 interacts with accessory device 180, as described above. Protruding portions of accessory device 180 may contact and be deflected by ramped surface 342 or otherwise directed so that accessory device 180 does not snag on elevator 300 (e.g., as accessory device 180 is moved proximally). As compared with elevator 200, accessory device 180 may contact and be deflected by ramped surface 342 of elevator 300 and may contact and be deflected by a portion of elevator 200 where ramped surface 242 and distalmost edge 252 meet.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. An elevator for a medical device, comprising:
a proximal end portion;
a distal end portion; and
a guide surface for contacting an accessory device, wherein the guide surface extends between the proximal end portion and the distal end portion;
wherein the distal end portion includes a curved distal surface, configured such that a protruding portion of the accessory device deflects off of the distal surface as the protruding portion is moved proximally.

2. The elevator of claim 1, wherein a central portion of the distal surface is longitudinally recessed with respect to side portions of the distal surface.

3. The elevator of claim 1, further comprising a back surface opposite the guide surface, wherein the curved distal surface extends between the guide surface and the back surface, wherein a central portion of the distal surface has a smaller thickness than side portions of the distal surface, wherein the thickness extends between the guide surface and the back surface.

4. The elevator of claim 1, wherein the distal surface intersects the guide surface at an edge, wherein a central portion of the edge extends farther distally than side portions of the edge.

5. The elevator of claim 4, further comprising a back surface, wherein the edge is a first edge, wherein the distal surface extends between the guide surface and the back surface, and wherein the distal surface intersects the back surface at a second edge, wherein side portions of the second edge extend farther distally than a central portion of the second edge.

6. The elevator of claim 1, wherein the distal surface has a hyperboloid shape.

7. The elevator of claim 1, wherein the distal surface extends a uniform amount in a distal direction across a width of the distal end portion.

8. The elevator of claim 1, wherein the distal surface is distal to an entirety of the guide surface.

9. The elevator of claim 1, wherein a central portion of the guide surface extends further in a distal direction than side portions of the guide surface.

10. The elevator of claim 1, wherein the distal end portion further includes a ramped surface, wherein the distal surface is a distal end of the ramped surface.

11. The elevator of claim 10, further comprising a back surface opposite the guide surface, wherein the ramped surface tapers toward the back surface moving in a distal direction.

12. The elevator of claim 11, wherein the ramped surface extends distally from a distal end of the guide surface.

13. The elevator of claim 1, wherein a straight line extends along an entire width of the distal surface.

14. The elevator of claim 1, wherein the distal surface is configured to inhibit the protruding portion of the accessory device from snagging on the elevator.

15. The elevator of claim 1, wherein the distal surface is configured to deflect off of the distal surface toward the guide surface as the protruding portion of the accessory device is moved proximally.

16. An elevator for a medical device, comprising:
a proximal end portion;
a distal end portion; and
a guide surface for contacting an accessory device, wherein the guide surface extends between the proximal end portion and the distal end portion;
wherein the distal end portion includes a curved distal surface, wherein the curved distal surface is thinner at a central portion of the curved distal surface than on sides of the curved distal surface.

17. The elevator of claim 16, wherein the central portion of the curved distal surface is proximal of the sides of the curved distal surface.

18. An elevator for a medical device, comprising:
a guide surface for contacting an accessory device;
a back surface, opposite the guide surface; and
a ramped distal surface extending distally from the guide surface toward the back surface, wherein a distalmost edge of the ramped distal surface is distal to an entirety of the guide surface.

19. The elevator of claim 18, wherein a straight line extends along the distalmost surface, between a first side of the distalmost edge and a second side of the distalmost edge.

20. The elevator of claim 19, wherein the distalmost edge is approximately perpendicular to a longitudinal axis of the guide surface.
